(19) United States
(12) Patent Application Publication
(10) Pub. No.: US 2007/0083125 A1
(43) Pub. Date: Apr. 12, 2007

(54) Apparatus and method of measuring biological information

(75) Inventors: Kazushige Ouchi, Kanagawa (JP); Takuji Suzuki, Kanagawa (JP); Akihisa Moriya, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Minato-ku (JP)

(21) Appl. No.: 11/512,241

(22) Filed: Aug. 30, 2006

(30) Foreign Application Priority Data
Aug. 31, 2005 (JP) 2005-252656

(51) Int. Cl. A61B 5/02

(52) U.S. Cl. 600/483

(57) ABSTRACT

A biological information measuring apparatus with a pillow shape having a convex portion to be contacted with a cervical region of a user, the apparatus includes a first measuring unit arranged on the convex portion measuring biological information about the user in a sleeping condition; a second measuring unit arranged in a position other than the convex portion measuring biological information about the user in the sleeping condition; a selecting unit selecting any one of the first and the second measuring unit based on the measured results of the first and second measuring unit; and a heart beat detecting unit detecting heart beats of the user based on the measured result of the first or the second measuring unit selected by the selecting unit.

### CROSS-REFERENCE TO RELATED APPLICATIONS

This application is based upon and claims the benefit of priority from the prior Japanese Patent Application No. 2005-252656, filed on Aug. 31, 2005; the entire contents of which are incorporated herein by reference.

### BACKGROUND OF THE INVENTION

1. Field of the Invention

The present invention relates to a pillow-shaped biological information measuring apparatus having a convex portion to be contacted with a cervical region of a user, and a biological information measuring method of measuring biological information about the user using the biological information measuring apparatus.

2. Description of the Related Art

Conventionally, a method of measuring a sleeping condition is generally all-night polygraph examination. In this examination, a plurality of sensors such as an electroencephalographic sensor, an electrocardiographic sensor, an electromyographic sensor, a respiration sensor and a sensor of saturated oxygen concentration in blood (SpO2) are attached to a user, so that a sleeping condition is known. This requires three-daysandtwo-nights examination in special facilities such as hospital, and thus this becomes a burden both on patients and doctors.

A mat-type sensor or the like has been, therefore, attempted to be arranged on a lower part of a user's body so as to prognose a sleeping condition easily. In such attempt, a pressure sensor or the like captures a change in pressure due to respiration, heart beat and body movement, and the change in pressure is utilized to prognose the sleeping condition. The sleeping condition is prognosed based on a fluctuation in a heart beat or based on activity of an automatic nerve system which can be known from a heart beat interval.

Meanwhile, as a smaller and more convenient constitution than the mat-type sensor, a similar sensor is arranged on a pillow so that the sleeping condition is prognosed. For example, a pillow into which a piezoelectric sheet is provided and which detects rapid eye movement (REM) sleep is proposed (JP-A No. 4-256732). Further, a sleeping condition is determined based on biological information measured by a mat-type sensor provided on an upper surface of a pillow and the pillow is deformed so as to obtain comfortable sleeping, is proposed (JP-A No. 2004-113329).

In order to prognose the sleeping condition in detail to some extent without measuring brain wave, it is considered to detect a heart beat so as to use a heart rate or an index of the natural nerve system obtained by frequency analysis of a beat peak-to-peak interval. With the mat-type sensor arranged on the pillow, however, a contact position, a contact portion and a contact state of a body to the pillow change due to a change in posture such as a dorsal position and a lateral position. In such a mat-type sensor, therefore, it is difficult to measure the heart beat stably through all night.

When an area of the sensor is increased in order to widen the contact portion, measuring sensitivity is deteriorated, and thus accurate measurement becomes difficult.

### SUMMARY OF THE INVENTION

According to one aspect of the present invention, a biological information measuring apparatus with a pillow shape having a convex portion to be contacted with a cervical region of a user, the apparatus includes a first measuring unit arranged on the convex portion measuring biological information about the user in sleeping condition; a second measuring unit arranged in a position other than the convex portion measuring biological information about the user in the sleeping condition; a selecting unit selecting any one of the first measuring unit and the second measuring unit based on the measured result of the first measuring unit and the measured result of the second measuring unit; and a heart beat detecting unit detecting heart beats of the user based on the measured result of the first measuring unit or the second measuring unit selected by the selecting unit.

According to another aspect of the present invention, a method of measuring biological information by using a biological information measuring apparatus with a pillow shape having a convex portion to be contacted with a cervical region of a user, the method includes measuring biological information about the user in a sleeping condition by a first measuring unit arranged on the convex portion; measuring biological information about the user in the sleeping condition by a second measuring unit arranged in a position other than the convex portion; selecting any one of the first measuring unit and the second measuring unit based on the measured result of the first measuring unit and the measured result of the second measuring unit; and detecting heart beats of the user based on the measured result of the selected any one of the first measuring unit and the second measuring unit.

### BRIEF DESCRIPTION OF THE DRAWINGS

FIG. 1 is a diagram illustrating an entire constitution of a biological information measuring apparatus 1 according to the present embodiment;

FIG. 2 is a diagram illustrating one example of a waveform of biological information detected by a first sensor 102 or a second sensor 104;

FIG. 3A is a diagram illustrating a waveform of each information separated from the biological information shown in FIG. 2;

FIG. 3B is a diagram illustrating a waveform of each information separated from the biological information shown in FIG. 2;

FIG. 4A is a diagram when a pillow 10 is viewed from a side surface;

FIG. 4B is a diagram when the pillow 10 is viewed from a top surface;

FIG. 5A is a diagram illustrating a waveform of the biological information detected by the first sensor 102 in a dorsal position;

FIG. 5B is a diagram illustrating a correlation between the biological information detected by the first sensor 102 in the dorsal position and a test pattern with respect to the biological information;

FIG. 6A is a diagram illustrating a waveform of biological information detected by the first sensor 102 in a lateral position;

FIG. 6B is a diagram illustrating a correlation coefficient of the biological information detected by the first sensor 102 in the lateral position;

FIG. 7A is a diagram illustrating a waveform of biological information detected by the second sensor 104 in the dorsal position;

FIG. 7B is a diagram illustrating a correlation between the biological information detected by the second sensor 104 in the dorsal position and a test pattern with respect to the biological information;

FIG. 8A is a diagram illustrating a waveform of biological information detected by the second sensor 104 in the lateral position;

FIG. 8B is a diagram illustrating a correlation coefficient of the biological information detected by the second sensor 104 in the lateral position;

FIG. 9 is a diagram illustrating a detailed constitution of the first sensor 102;

FIG. 10 is a flowchart illustrating a biological information measuring process in the biological information measuring apparatus 1;

FIG. 11 is a flowchart illustrating a detailed process in a measuring accuracy evaluating process (step S102) described in FIG. 10;

FIG. 12 is a diagram for more specifically explaining the measuring accuracy evaluating process (step S102);

FIG. 13 is a diagram for more specifically explaining the measuring accuracy evaluating process (step S102);

FIG. 14 is a flowchart illustrating a detailed process in a various information detection process (step S108) shown in FIG. 10;

FIG. 15 a flowchart illustrating a detailed process in a sensor selecting process (step S120) shown in FIG. 10;

FIG. 16 is a diagram illustrating a display example of an analyzed result of a sleeping condition;

FIG. 17 is a diagram illustrating a hardware structure of the biological information measuring apparatus 1 according to the present embodiment;

FIG. 18 is a diagram illustrating a range in which the second sensor 104 can be provided;

FIG. 19 is a diagram illustrating one example of the arrangement of the second sensor 104;

FIG. 20 is a diagram illustrating one example of the arrangement of the second sensor 104;

FIG. 21 is a diagram illustrating one example of the arrangement of the second sensor 104;

FIG. 22 is a diagram illustrating an appearance of the pillow 10 according to a second modified example; and

FIG. 23 is a diagram illustrating an appearance of the pillow 10 according to a third modified example.

### DETAILED DESCRIPTION OF THE INVENTION

A biological information measuring apparatus and a biological information measuring method according to an embodiment of the present invention are explained in detail below with reference to the drawings. The present invention is not limited to the embodiment.

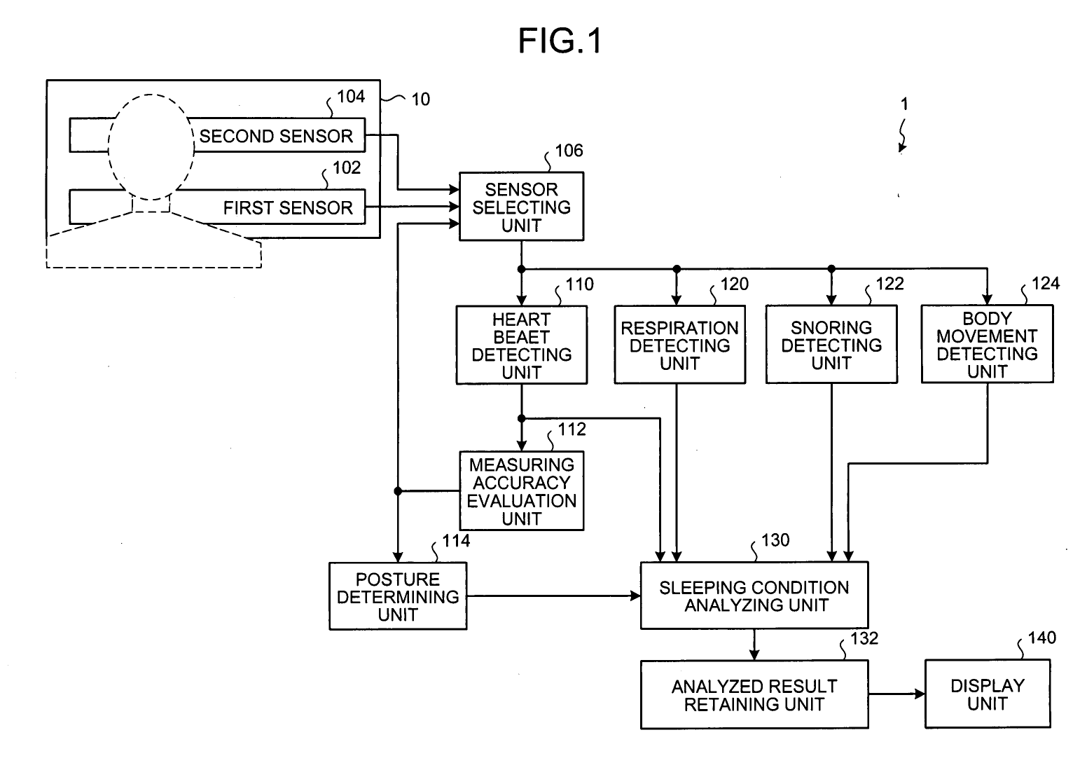

FIG. 1 is a diagram illustrating an entire constitution of the biological information measuring apparatus 1 according to an embodiment. The biological information measuring apparatus 1 has a first sensor 102 and a second sensor 104 which are built in a pillow 10, a sensor selecting unit 106, a heart beat detecting unit 110, a measuring accuracy evaluation unit 112, a posture determining unit 114, a respiration detecting unit 120, a snoring detecting unit 122, a body movement detecting unit 124, a sleeping condition analyzing unit 130, an analyzed result retaining unit 132, and a display unit 140.

The first sensor 102 and the second sensor 104 detect biological information during the time when a user is sleeping. Here, the biological information includes information about pulsation on a portion which contacts with the sensor, such as the occipital region of the head, and pulsation on arteria carotis. The first sensor 102 and the second sensor 104 in this embodiment correspond to a first measuring unit and a second measuring unit, respectively.

The sensor selecting unit 106 selects any one of the first sensor 102 and the second sensor 104 as a sensor which should acquire biological information to be utilized for a sleeping condition analyzing process, mentioned later. The heart beat detecting unit 110 acquires the biological information detected by the sensor selected by the sensor selecting unit 106. The heart beat detecting unit 110 separates heart beat information from the biological information based on a frequency component and a signal level. Here, the heart beat information is a signal which changes according to the heart beat of the user. Specifically, the heart beat information is separated from the biological information by BPF (band-pass filter) with cut-off frequency of about 1 Hz to 5 Hz.

The respiration detecting unit 120 separates respiration information from the biological information based on a frequency component and a signal level. Here, the respiration information is a signal which changes according to respiration of the user. The respiration information is separated from the biological information by LPF (low-pass filter) with cut-off frequency of about 1 Hz.

The snoring detecting unit 122 separates snoring information from the biological information based on a frequency component and a signal level. Here, the snoring information is a signal which changes according to snoring of the user. The snoring information is separated from the biological information by HPF (high-pass filter) with cut-off frequency of about 5 Hz.

The body movement detecting unit 124 detects body movement information from the biological information based on a frequency component and a signal level. Here, the body movement information is a signal which changes according to body movement of the user. For example, when a threshold of the signal level of the biological information is set and the signal exceeds the threshold, the waveform at this time is acquired as the body movement information.

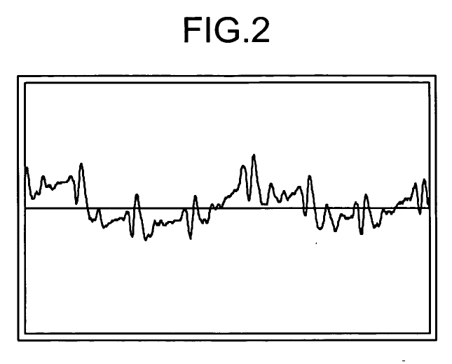

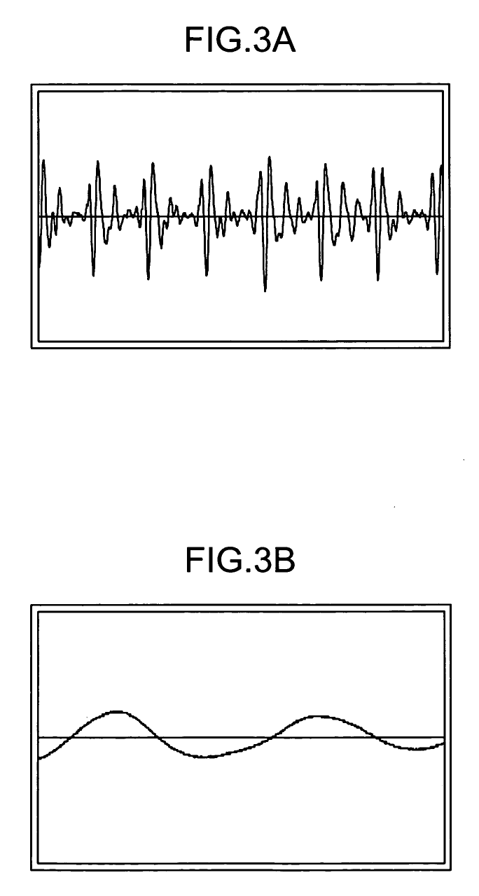

FIG. 2 is a diagram illustrating one example of a waveform of the biological information detected by the first sensor 102 or the second sensor 104. In the waveform of the biological information shown in FIG. 2, a plurality of signals are added. FIGS. 3A and 3B illustrate waveforms of each information separated from the biological information shown in FIG. 2. FIG. 3A is a diagram illustrating the heart beat information. FIG. 3B is a diagram illustrating the respiration information.

The measuring accuracy evaluation unit 112 acquires the heart beat information from the heart beat detecting unit 110, and evaluates measuring accuracy of the first sensor 102 or the second sensor 104 based on the heart beat information. The measuring accuracy evaluation unit 112 transmits the evaluated result to the posture determining unit 114 and the sensor selecting unit 106. More specifically, the measuring accuracy evaluation unit 112 previously retains a test pattern with respect to the heart beat information. It then calculates a correlation coefficient representing a correlation with the test pattern. The unit 112 evaluates the measuring accuracy based on the correlation coefficient.

The posture determining unit 114 determines a posture of the user based on the evaluated result obtained by the measuring accuracy evaluation unit 112. The postures, which can be determined, include a lateral position in which a right-left direction of the user's head portion is parallel to a vertical direction of the pillow 10, and a dorsal position in which the right-left direction of the user's head portion is parallel to the right-left direction of the pillow 10.

The sleeping condition analyzing unit 130 acquires the heart beat information from the heart beat detecting unit 110. The unit 130 also acquires the respiration information from the respiration detecting unit 120. The unit 130 acquires the snoring information from the snoring detecting unit 122. The unit 130 acquires the body movement information from the body movement detecting unit 124. The unit 130 acquires the posture determination result from the posture determining unit 114. The sleeping condition analyzing unit 130 analyzes the sleeping condition based on these information.

Specifically, the sleeping condition analyzing unit 130 specifies the degree of activity of the automatic nerve system obtained by frequency-analyzing a heart rate or the beat peak-to-peak interval from the heart beat information. Further, the sleeping condition analyzing unit 130 analyzes the sleeping condition such as REM sleeping or non-REM sleeping using the respiration information, snoring information, body movement information, posture information and the like.

The sleeping condition analyzing unit 130 does not have to use all the information as long as at least the heart beat information in the sleeping condition analysis is used.

The analyzed result retaining unit 132 retains the analyzed result of the sleeping condition obtained by the sleeping condition analyzing unit 130. The display unit 140 displays the analyzed result of the sleeping condition retained in the analyzed result retaining unit 132 in a form of a graph, numerical values and the like. The display unit 140 may display the analyzed result of the sleeping condition in real time, or in another manner, it may display the analyzed result according to user's request.

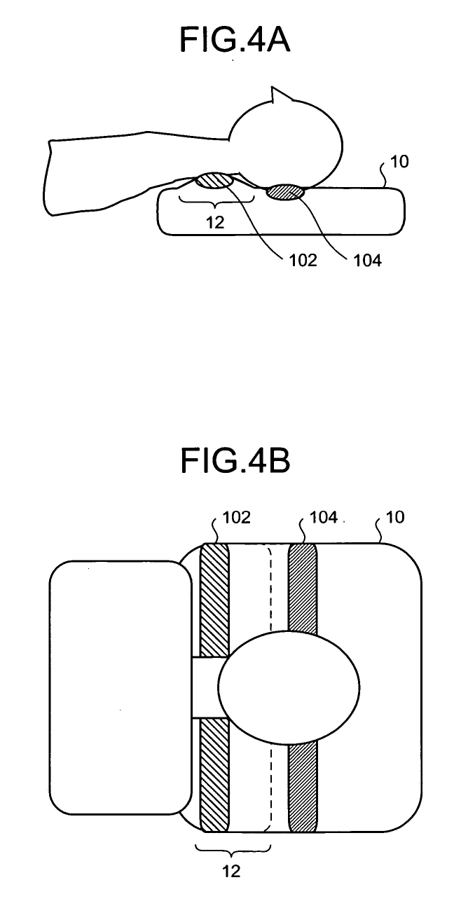

FIG. 4A is a diagram when the pillow 10 is viewed from the side surface. FIG. 4B is a diagram when the pillow 10 is viewed from the top surface. The pillow 10 has a convex portion 12 which should be into contact with a cervical region of the user. The convex portion 12 is provided with the first sensor 102.

When the first sensor 102 is provided to the convex portion 12, the first sensor 102 can detect pulsation of the artery at the cervical region of the user. Since the first sensor 102 is formed on the convex portion 12, even if the user is in the lateral position, the sensor can satisfactorily keep the contact state with the user.

The second sensor 104 is provided to a region other than the convex portion 12. More specifically, the second sensor 104 is provided in a position which comes into contact with the occipital region of the user's head. As a result, the second sensor 104 can detect the pulsation at the occipital region of the user's head as biological information.

The first sensor 102 and the second sensor 104 are formed in a lateral direction of the pillow uniformly from the right end to the left end. Since the first sensor 102 and the second sensor 104 are formed from the right end to the left end of the pillow 10, even if the user moves to the right-left direction at the time of rolling over, the biological information can be detected accurately.

The width of the first sensor 102 and the second sensor 104 in the vertical direction of the pillow is preferably not more than 100 mm. The reason for this is as follows. That is to say, when the width in the vertical direction of the pillow is too large, biological information in a plurality of paths is occasionally detected in such a manner that both a pulse wave of the arteria carotis and pulsation of the head portion are detected. As a result, the accurate biological information cannot be detected.

When the body movement of the use in the sleeping condition is-taken into consideration, the pillow 10 should be formed widely in the lateral direction. Meanwhile, when the volume of the first sensor 102 and the second sensor 104 is too large, the detecting sensitivity is deteriorated. According to these, the width of the pillow 10 in the lateral direction is increased, whereas the width in the vertical direction is decreased.

The convex portion 12 of the pillow 10 is formed so as to fit the shape from the cervical region to the head portion of the user. For this reason, it is considered that the head portion is often put on the position where the cervical region comes into contact with the convex portion 12. That is to say, when the convex portion 12 is provided, even if the user rolls over in the sleeping condition, the position of the head portion can be fixed in the vertical direction of the pillow to some extent. As a result, the contact states of the first sensor 102 and the second sensor 104 can be retained satisfactorily.

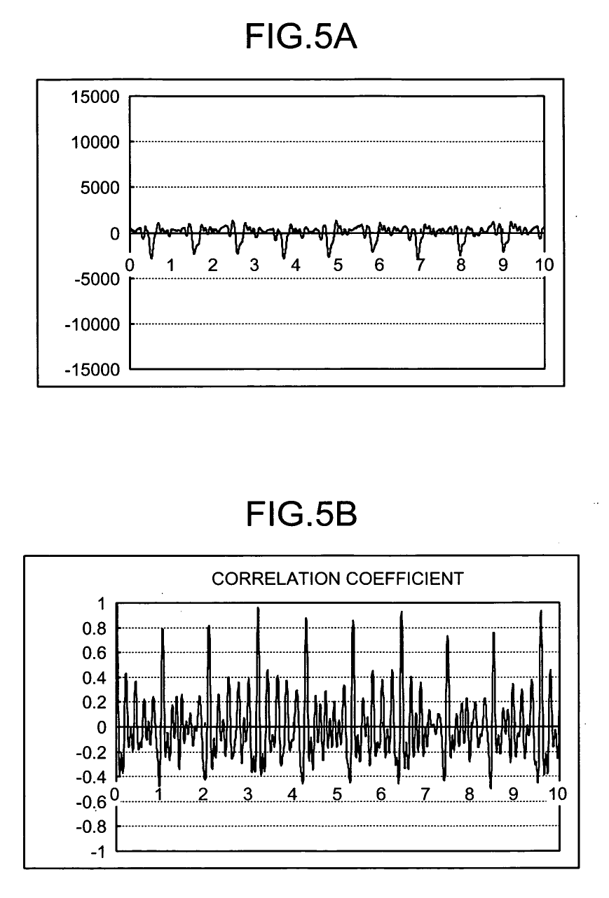

The first sensor 102 is applicable for the measurement in the lateral position. The second sensor 104 is applicable for the measurement in the dorsal position. These sensors are explained in detail with reference to FIGS. 5A to 8B. FIG. 5A is a diagram illustrating a waveform of the biological information detected by the first sensor 102 in the dorsal position. FIG. 5B is a diagram illustrating a correlation coefficient showing a correlation between the biological information detected by the first sensor 102 in the dorsal position and a test pattern with respect to the biological information.

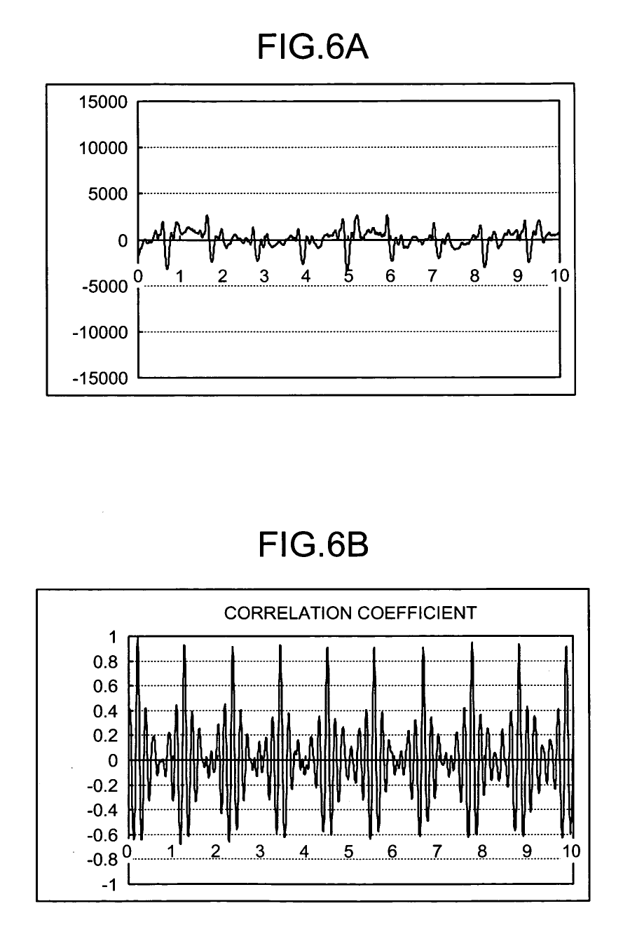

FIG. 6A is a diagram illustrating a waveform of the biological information detected by the first sensor 102 in the lateral position. FIG. 6B is a diagram illustrating a correlation coefficient of the biological information detected by the first sensor 102 in the lateral position. As shown in these drawings, the correlation coefficient in the lateral position is stable at relatively high values. On the contrary, the correlation coefficient in the dorsal position largely disperses and is unstable. That is to say, the first sensor 102 is applicable for the detection of the biological information in the lateral position.

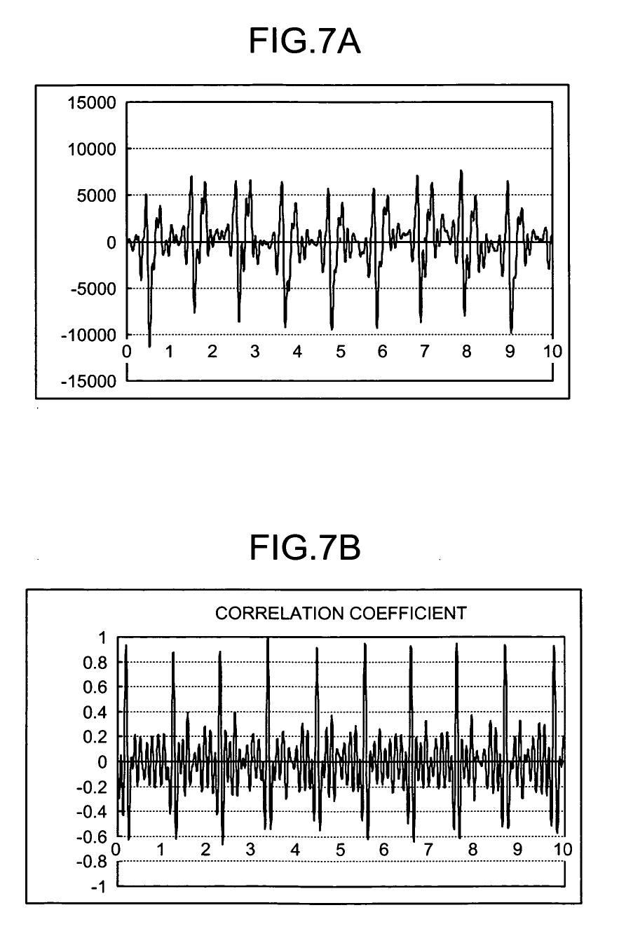

Meanwhile, FIG. 7A is a diagram illustrating a waveform of the biological information detected by the second sensor 104 in the dorsal position. FIG. 7B is a diagram illustrating a correlation coefficient showing a correlation between the biological information detected by the second sensor 104 in the dorsal position and a test pattern with respect to the biological information.

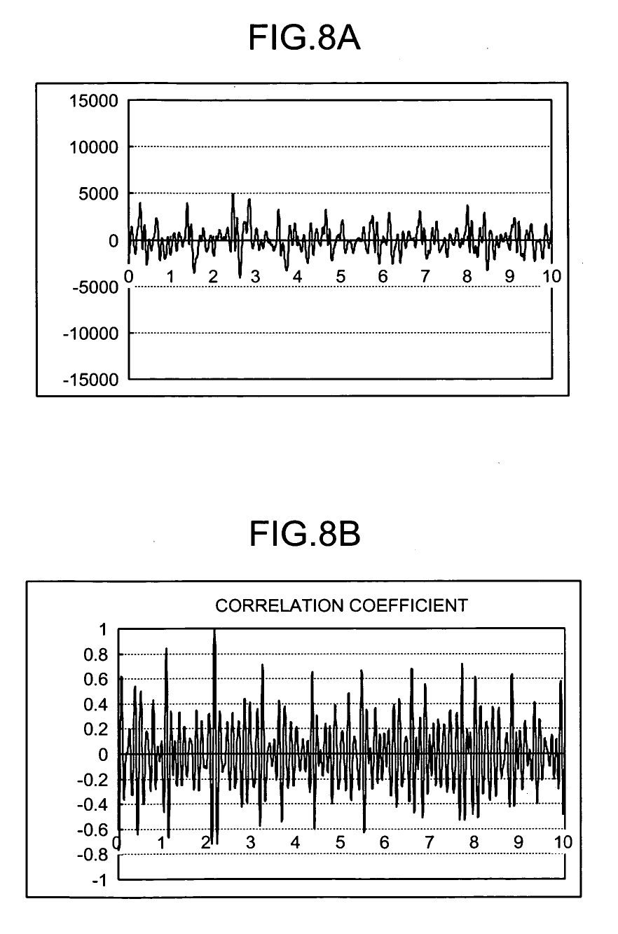

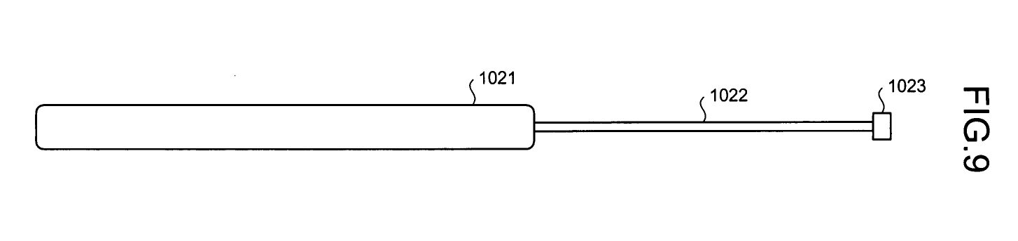

FIG. 8A is a diagram illustrating a waveform of the biological information detected by the second sensor 104 in the lateral position. FIG. 8B is a diagram illustrating a correlation coefficient of the biological information detected by the second sensor 104 in the lateral position. As shown in the drawings, the correlation coefficient in the dorsal position is stable. On the contrary, the correlation coefficient in the lateral position largely disperses and is unstable. That is to say, the second sensor 104 is applicable for the detection of the biological information in the dorsal position.

The detection accuracy of any one of the first sensor 102 and the second sensor 104 is higher than that of the other one according to the postures of the user in the sleeping condition. For this reason, the posture of the user in the sleeping condition can be determined based on the detection accuracies of the two sensors.

FIG. 9 is a diagram illustrating a detailed constitution of the first sensor 102. The first sensor 102 has an air mat 1021 built into the pillow 10, a rubber tube 1022 formed integrally with the air mat 1021, and a pressure sensor 1023 that detects a change in the pressure in the air mat 1021 via the rubber tube 1022.

As another example, the pressure sensor 1023 may be connected directly to the air mat 1021 so as to detect a change in the pressure not via the rubber tube 1022.

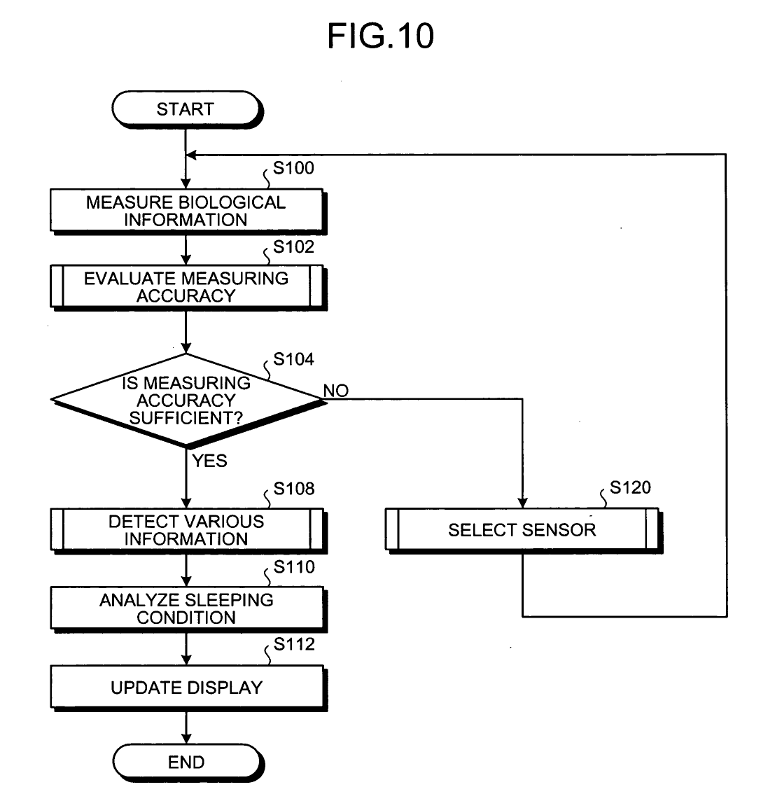

FIG. 10 is a flowchart illustrating a biological information measuring process in the biological information measuring apparatus 1. The sensor selected from the first sensor 102 and the second sensor 104 by the sensor selecting unit 106 measures biological information (step S100). When any sensor is not selected at the time of actuating the biological information measuring apparatus 1, the first sensor 102 may measure the biological information as a default setting. In another example, the sensor may be selected by a sensor selecting process (step S120), mentioned later.

Then, the measuring accuracy evaluation unit 112 evaluates measuring accuracy in the selected sensor (step S102). Specifically, the unit 112 evaluates the measuring accuracy based on the correlation coefficient of the waveform of the heart beat obtained by the heart beat detecting unit 110.

When the measuring accuracy is sufficient (Yes at step S104), the currently selected sensor is determined as applicable, and a sleeping condition is analyzed based on the biological information measured by the selected sensor. Specifically, various information is obtained (step S108). Then, the sleeping condition analyzing unit 130 determines the sleeping condition based on the obtained information (step S110), and displays the result (step S112). The biological information measuring process is completed here.

On the other hand, the measuring accuracy is not sufficient at step S104 (No at step S104), the sensor selecting unit 106 selects the applicable sensor based on the measured result of the biological information in the respective sensors by the measuring accuracy evaluation unit 112 (step S120).

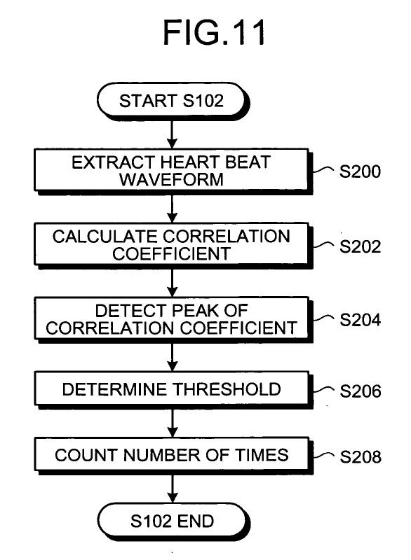

FIG. 11 is a flowchart illustrating a detailed process in the measuring accuracy evaluating process (step S120) explained with reference to FIG. 10. First, the measuring accuracy evaluation unit 112 picks up a signal of a frequency component (for example, 1 Hz to 5 Hz) corresponding to the heart beat from the biological information measured for predetermined time period, namely, for unit time (step S200). The unit 112 calculates the correlation coefficient between a template waveform previously retained in the measuring accuracy evaluation unit 112 and the pick-up signal (step S202).

The measuring accuracy evaluation unit 112 retains one template waveform which is used for the biological information measured by the first sensor 102 and one template waveform which is used for the biological information measured by the second sensor 104. The measuring accuracy evaluation unit 112 calculates the correlation coefficient using the corresponding templates.

In another example, only one template waveform may be retained. In this case, the correlation coefficient is calculated by utilizing the same template waveform as to both the biological information measured by the first sensor 102 and the biological information measured by the second sensor 104.

In another example, a plurality of template waveforms to be used for the biological information measured by the first sensor 102 may be retained, and a plurality of template waveforms to be used for the biological information measured by the second sensor 104 may be retained. In this case, it is preferable that reproducibilities in the plural template waveforms are evaluated, and the template waveform with the highest reproducibility is used.

In another example, the template waveform retained in the measuring accuracy evaluation unit 112 may be dynamically created by the measuring accuracy evaluation unit 112. Specifically, for example, after the template is created, the first heart beat waveform within plural heart beat waveforms measured for a prescribed time period may be used. In another example, some candidate template waveforms are extracted from the plural heart beat waveforms, and the template waveform with the highest reproducibility may be used.

Next, the measuring accuracy evaluation unit 112 detects a peak of the correlation coefficient (step S204). The measuring accuracy evaluation unit 112 determines whether the value of the detected peak exceeds a preset threshold (step S206), and counts the number of peaks which exceeds the threshold (step S208). The measuring accuracy evaluating process (step S102) is completed here.

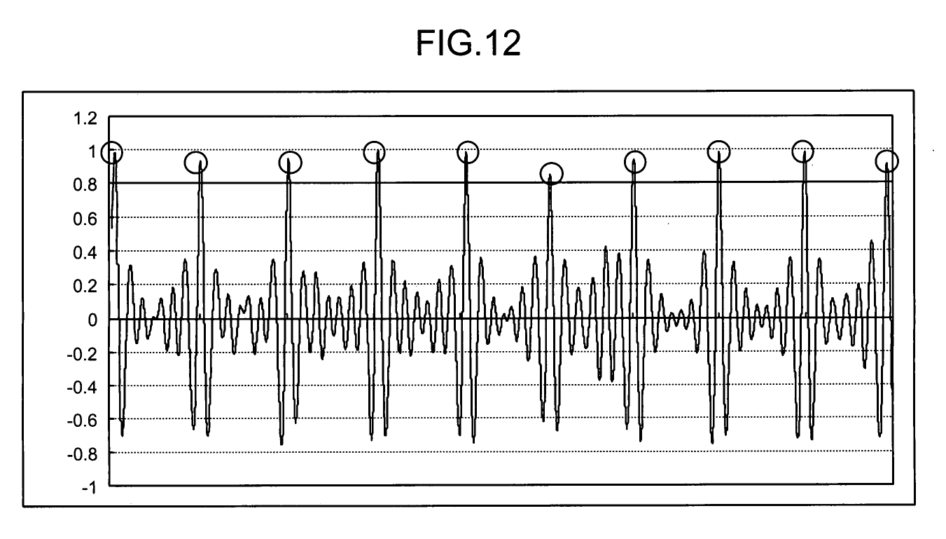

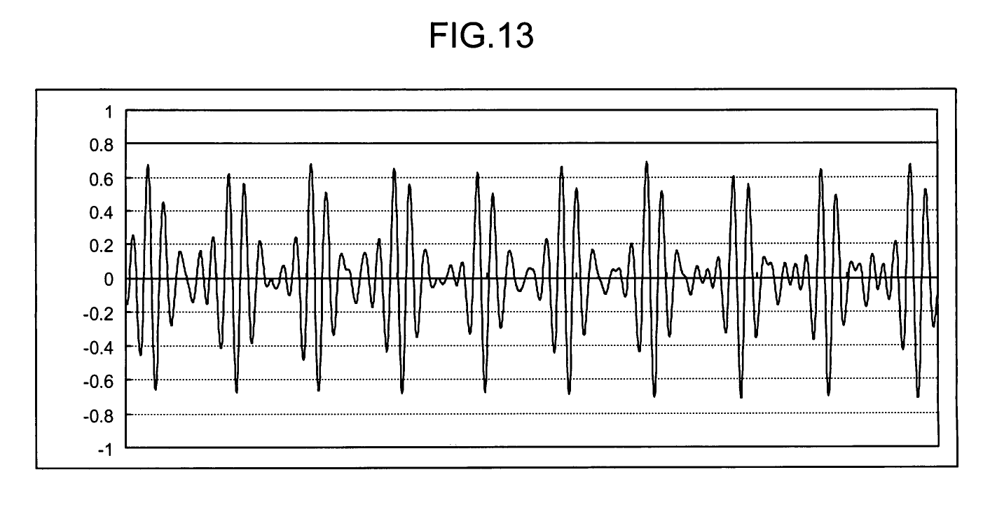

FIGS. 12 and 13 are diagrams for specifically explaining the measuring accuracy evaluating process (step S102). FIG. 12 is a diagram illustrating a graph of the correlation coefficient showing the peaks which exceed the threshold. In the example of FIG. 12, the threshold is set to 0.8. In the example shown in FIG. 12, all the peaks exceed the threshold. At step S208, therefore, the number of all the peaks is counted.

Meanwhile, FIG. 13 is a diagram illustrating a graph of the correlation coefficient which does not include the peaks exceeding the threshold. The threshold in the example shown in FIG. 13 is 0.8. In the example shown in FIG. 13, all the peaks are less than the threshold. In this case, the counting number is “0”.

When the number of the peaks more than the threshold counted at step S208 is not less than a predetermined number, the measuring accuracy evaluation unit 112 evaluates that the measuring accuracy is satisfactory. Meanwhile, when the number of the peaks is less than the predetermined number, the unit 112 evaluates that the measuring accuracy is defective. The predetermined number may be, for example, a number which is 90% of the number of all the peaks for unit time.

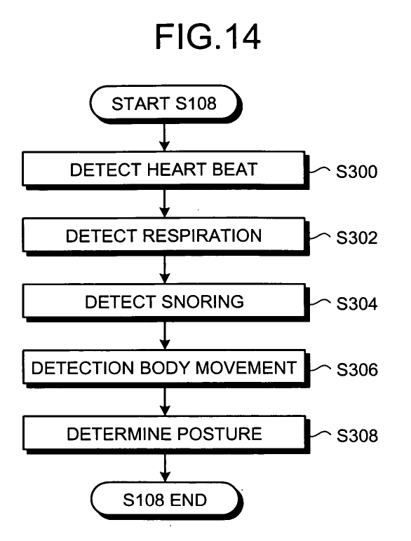

FIG. 14 is a flowchart illustrating the detailed process in the various information detection process (step S108) shown in FIG. 10. The heart beat detecting unit 110 picks up a signal of a frequency component (for example, 1 Hz to 5 Hz) corresponding to the heart beat from the biological information (step S300). The respiration detecting unit 120 picks up a signal of a frequency component corresponding to the respiration (step S302). The snoring detecting unit 122 picks up a signal of a frequency component corresponding to the snoring (step S304). The body movement detecting unit 124 picks up a body movement signal (step S306). The posture determining unit 114 determines posture (step S308) The various information detection process is completed here.

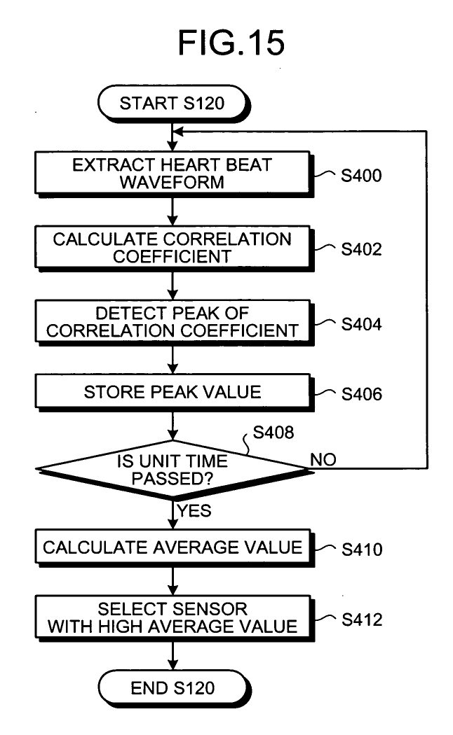

FIG. 15 is a flowchart illustrating the detailed process in the sensor selecting process (step S120) shown in FIG. 10. The heart beat information is extracted from the biological information (step S400). The correlation coefficient is calculated based on the heart beat information and the template waveform previously retained in the measuring accuracy evaluation unit 112 (step S402). This process is similar to the correlation coefficient calculating process (step S202) explained with reference to FIG. 11.

Peaks of the correlation coefficient is detected (step S404), and the peak values are stored (step S406). These steps are executed on the biological information measured during the unit time (step S408). An average value of the plural peak values measured and stored for the unit time is calculated (step S410).

These steps are executed on the biological information measured by the first sensor 102 and the biological information measured by the second sensor 104. The sensor selecting unit 106 selects a sensor which calculates the larger average value (step S412). The sensor selecting process (step S120) is completed here.

In this embodiment, the sensor is selected based on the average values of the peaks calculated from the biological information measured by the first sensor 102 and the biological information measured by the second sensor 104. The selecting method is not, however, limited to this method as long as the sensor is selected based on the peak values.

In another example, a sensor may be selected based on standard deviations of the peaks instead of the average values of the peaks. In another example, a sensor may be selected based on the number of peaks exceeding a predetermined threshold.

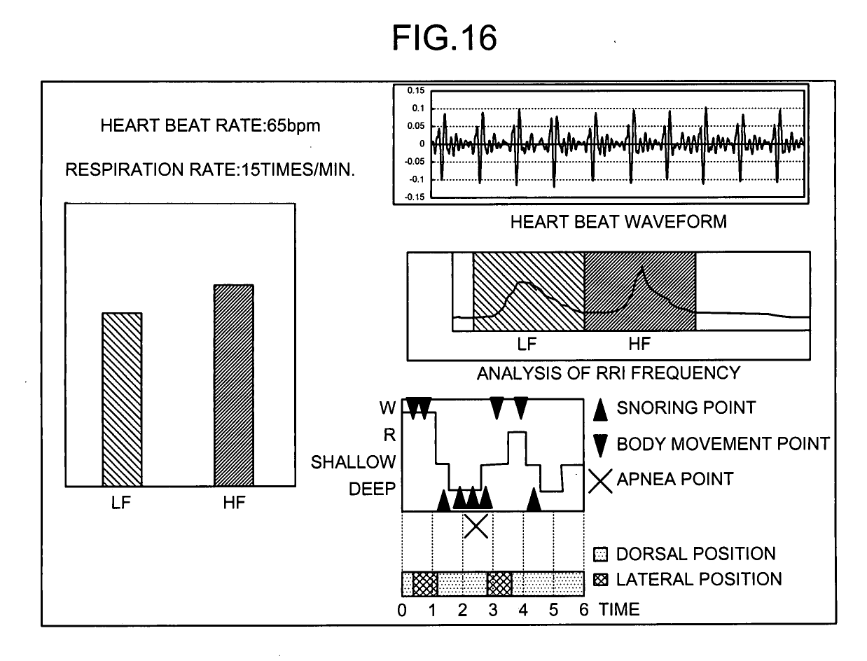

FIG. 16 is a diagram illustrating a display example of the sleeping condition analyzed result obtained by the above process. As shown in FIG. 16, the display unit 140 displays sleeping depth at each time during sleeping, apnea point showing the time at which the apnea state is detected, body movement point showing the time at which a body movement is detected, and the like. Further, the display unit 140 displays whether a user is in the dorsal position or the lateral position at each time. The display unit 140 displays the heart rate, the respiratory rate, a waveform of heart beat measured at each time, and the like.

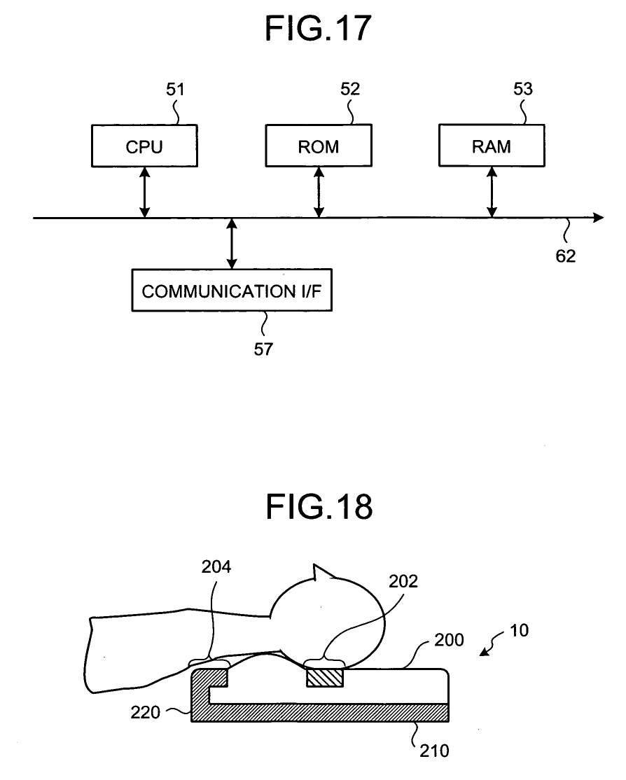

FIG. 17 is a diagram illustrating a hardware structure of the biological information measuring apparatus 1 according to this embodiment. The biological information measuring apparatus 1 provides a ROM 52 which stores a biological information measuring program or the like for executing the processes after the sensor selecting process in the biological information measuring apparatus 1, a CPU 51 which controls the respective units of the biological information measuring apparatus 1 according to the program in the ROM 52, a RAM 53 which stores various data necessary for controlling the biological information measuring apparatus 1, a communication I/F 57 which is connected to a network so as to make a communication, and a bus 62 which connects the respective units.

The above mentioned biological information measuring program in the biological information measuring apparatus 1 may be provided in a state where it is recorded on a computer readable recording medium with a file of installable or executable format, such as a CD-ROM, a floppy (registered trademark) disc (FD), a DVD, or the like.

In this case, the biological information measuring program is read from the recording medium so as to be executed in the biological information measuring apparatus 1. As a result, the program is loaded on a main storage device, and the respective units explained in the software structure are created on the main storage device.

The biological information measuring program of this embodiment may be stored in a computer connected to a network such as internet, and may be provided by performing a download via the network.

The present invention is explained above based on the embodiment, but the above embodiment can be changed and modified variously.

In a first modified example, the pillow 10 according to the embodiment provides the first sensor 102 at the convex portion 12 thereof, and the second sensor 104 is provided to the position which should be come into contact with the occipital region of the user's head. The position where the second sensor 104 is formed may be, however, a position which is other than the convex portion 12 in the pillow 10 and comes into contact with the user, and thus the position is not limited to the embodiment.

FIG. 18 is a diagram illustrating a range where the second sensor 104 can be provided. The second sensor 104 may be formed in any one of regions 202, 204, 210 and 220 shown by slanted lines in FIG. 18. As shown in FIG. 18, on a surface of the pillow 10 which comes into contact with the user, namely, the surface 200, the second sensor 104 may be formed on the position 204 which comes into contact with shoulders other than the position 202 which comes into contact with the occipital region of the user's head.

The second sensor 104 may be formed in any position of the rear surface 210 of the pillow 10. The second sensor 104 may be formed in any position of the lower side surface 220 in the pillow 10. In any cases, it is desirable that after user's movement toward the lateral direction is considered, the second sensor 104 is formed from the right end to the left end of the pillow.

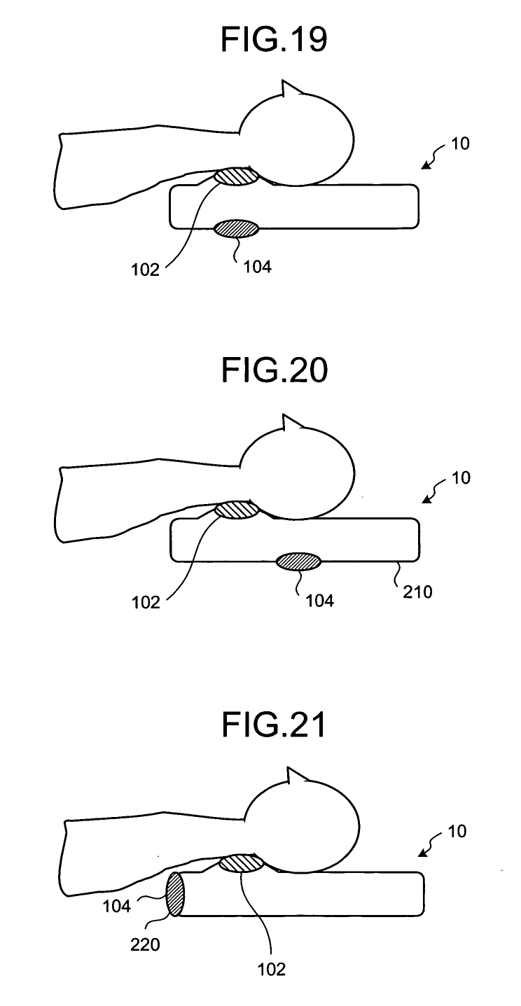

FIGS. 19 to 21 are diagrams illustrating examples of arrangement of the second sensor 104. In the example shown in FIG. 19, the second sensor 104 is formed on a position of the rear surface 210, which is opposed to the surface where the first sensor 102 is formed. In the example shown in FIG. 20, the second sensor 104 is formed on a position of the rear surface 210 which is opposed to the surface which comes into contact with the occipital region of the user's head. In the example shown in FIG. 21, the second sensor 104 is formed on the side surface 220.

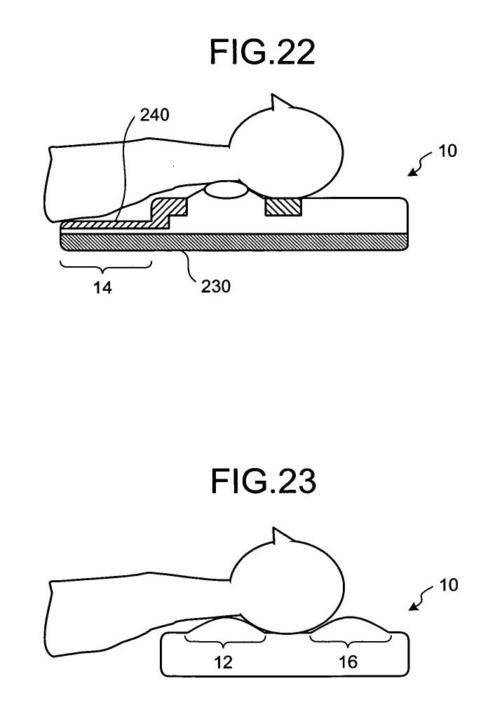

FIG. 22 is a diagram illustrating an appearance of the pillow 10 according to a second modified example. The pillow 10 of this example has a pad portion 14 which extends from the pillow main body. The pad portion 14 is arranged in a portion to be contacted with a user's blade bone. In this case, the second sensor 104 may be formed in any position of the front surface 240 and the rear surface 230 of the pad portion 14. Also in this case, it is desirable that the second sensor 104 is formed from the right end to the left end of the pad portion 14.

FIG. 23 is a diagram illustrating an appearance of the pillow 10 according to a third modified example. The pillow 10 of this example has two convex portions 12 and 16. Since the pillow 10 of this example is formed with the additional convex portion 16, the position of the upper portion of the user's head in the vertical direction can be fixed. With this configuration, the contact position of the user in the vertical direction can be fixed to some extent.

The correlation coefficient is used for the evaluation of the measuring accuracy in the embodiment, but as a fourth modified example, the evaluation of the measuring accuracy is not limited to this, and thus an S/N ratio is detected based on the peaks of the heart beat included in the biological information measured by the sensor, and the measuring accuracy may be evaluated based on the S/N ratio.

In this embodiment, when the measuring accuracy in the selected sensor becomes insufficient, the sensor selecting process is executed, but as a fifth modified example, instead of this, the sensor selecting process may be executed at the time of every measurement or every time when the predetermined time period passes.

As mentioned above, according to the present invention, heart beats can be measured accurately regardless of postures of a user.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A biological information measuring apparatus with a pillow shape having a convex portion to be contacted with a cervical region of a user, comprising:
a first measuring unit arranged on the convex portion measuring biological information about the user in a sleeping condition;
a second measuring unit arranged in a position other than the convex portion measuring biological information about the user in the sleeping condition;
a selecting unit selecting any one of the first measuring unit and the second measuring unit based on the measured result of the first measuring unit and the measured result of the second measuring unit; and
a heart beat detecting unit detecting heart beats of the user based on the measured result of the first measuring unit or the second measuring unit selected by the selecting unit.

2. The apparatus according to claim 1, wherein the second measuring unit is arranged in a position to be contacted with the occipital region of a user's head.

3. The apparatus according to claim 1, wherein the second measuring unit is arranged in a position to be contacted with user's shoulders.

4. The apparatus according to claim 1, wherein the second measuring unit is provided on a same surface where the convex portion is formed.

5. The apparatus according to claim 1, wherein the second measuring unit is provided on a surface opposite to the surface where the convex portion is formed.

6. The apparatus according to claim 1 further comprising:
a pad portion to be contacted with a user's blade bone formed integrally with a pillow main body having the convex portion,
wherein the second measuring unit is provided to the pad portion.

7. The apparatus according to claim 1 further comprising:
a posture determining unit determining a posture of the user based on the measured result of the first measuring unit and the measured result of the second measuring unit; and
a sleeping condition determining unit determining a sleeping condition of the user based on the detected result of the heart beat detecting unit and the posture determination result of the posture determining unit.

8. The apparatus according to claim 1 further comprising:
a measuring accuracy detecting unit detecting measuring accuracy in any one of the first measuring unit and the second measuring unit selected by the selecting unit,
wherein the selecting unit selects any one of the first measuring unit and the second measuring unit when the measuring accuracy detected by the measuring accuracy detecting unit becomes smaller than a predetermined threshold.

9. The apparatus according to claim 1 further comprising:
a measuring accuracy detecting unit detecting measuring accuracy in the first measuring unit and measuring accuracy in the second measuring unit,
wherein the selecting unit selects the measuring unit with higher measuring accuracy detected by the measuring accuracy detecting unit from the first measuring unit and the second measuring unit.

10. A method of measuring biological information by using a biological information measuring apparatus with a pillow shape having a convex portion to be contacted with a cervical region of a user, comprising:
measuring biological information about the user in a sleeping condition by a first measuring unit arranged on the convex portion;
measuring biological information about the user in the sleeping condition by a second measuring unit arranged in a position other than the convex portion;
selecting any one of the first measuring unit and the second measuring unit based on the measured result of the first measuring unit and the measured result of the second measuring unit; and
detecting heart beats of the user based on the measured result of the selected any one of the first measuring unit and the second measuring unit.
